(19) United States
(12) Patent Application Publication
(10) Pub. No.: US 2016/0339131 A1
(43) Pub. Date: Nov. 24, 2016

(54) ODOR REMOVAL ASSEMBLY

(71) Applicant: MoJack Distributors, LLC, Wichita, KS (US)

(72) Inventor: Daniel V. Drake, Wichita, KS (US)

(21) Appl. No.: 15/219,072

(22) Filed: Jul. 25, 2016

Related U.S. Application Data
(63) Continuation-in-part of application No. 14/662,032, filed on Mar. 18, 2015, now Pat. No. 9,399,834.

(51) Int. Cl. A61L 2/20; A45C 5/03; A45C 13/10; A45C 3/00

(52) U.S. Cl. CPC A61L 2/202; A45C 3/001; A45C 5/03; A45C 13/103; A45C 2003/007; A61L 2202/11; A61L 2202/26

(57) ABSTRACT

An odor removal assembly comprising a container that, when open, allows a user to place items into the bag's interior, and when closed, prevents a user from placing items into the interior of the bag. The container may include a fastening mechanism for closing the container. The assembly includes an ozone generator that generates ozone and expels the ozone through a conduit that extends between an outlet of the ozone generator and the interior of the container. The assembly may also include second container configured for placement within the first container. In such embodiments, a user may utilize a portable bag for use with the odor removal assembly without having to carry around the actual ozone generator. Rather, the user may insert the second container into the first in order to utilize the odor removal assembly.

### BACKGROUND

Removing odors from items such as clothing and other gear has become increasingly important to hunters, sportsmen, and to the general public. Accordingly, there is a need for improved devices that make removing odors from these items more efficient. There is currently a particular need for removing odors from athletic gear such as athletic jerseys, pads, helmets, braces, shoes, athletic gear, and hunting gear. Various embodiments of the present odor removing ozone assembly recognize and address the foregoing considerations, and others, of prior art devices.

### SUMMARY

An assembly for removing odors from clothing or other items, according to various embodiments, comprises: a first container comprising a material that is at least partially ozone impervious and defining a first interior that is accessible via a selectively closeable opening; an ozone generator that is adapted to generate ozone gas and to expel the ozone gas from the ozone generator through an outlet; at least one conduit that extends in gaseous communication between the outlet of the ozone generator and the first interior of the first container; a fastening mechanism for selectively maintaining the selectively closeable opening in a closed orientation in which the fastening mechanism prevents access to the first interior via the selectively closeable opening; and a second container defining a second interior for at least temporarily storing one or more items for odor removal. In particular embodiments, the selectively closeable opening is in an open orientation in which the fastening mechanism does not prevent access to the first interior, a user may place the second container into the first interior of the first container via the selectively closeable opening. In still other embodiments, when the second container is in the first interior of the first container, the first interior is in gaseous communication with the second interior allowing for a transfer of at least some ozone gas from the first interior to the second interior.

A method of removing odors from one or more items, according to particular embodiments: (1) providing a first container comprising a material that is at least partially ozone impervious and defining a first interior that is accessible via a selectively closeable opening, wherein the first container comprises a fastening mechanism for selectively maintaining the selectively closeable opening in a closed orientation in which the fastening mechanism prevents access to the first interior via the selectively closeable opening; (2) providing an ozone generator that is adapted to generate ozone gas and to expel the ozone gas from the ozone generator through an outlet; (3) providing at least one conduit; (4) placing the at least one conduit such that the at least one conduit extends in gaseous communication between the outlet of the ozone generator and the first interior of the first container; (5) placing one or more items in an interior portion of a second container; (6) placing the second container within the first container such that the first interior is in gaseous communication with the second interior of the second container; (7) using the fastening mechanism to maintain the selectively closeable opening in the closed position; and (8) turning on the ozone generator to cause at least some ozone gas to expel into the first interior portion through the at least one conduit.

A method of removing odors from one or more items using an ozone generator, in some embodiments, comprises: (1) providing an ozone generator that is adapted to generate ozone gas and to expel the ozone gas from the ozone generator through an outlet; (2) providing at least one conduit; (3) placing the at least one conduit such that the at least one conduit extends in gaseous communication between the outlet of the ozone generator and a first interior of a rigid bin, the rigid bin comprising a locking mechanism and a lid and defining an interior chamber accessible via on opening, wherein the locking mechanism is configured to maintain the lid adjacent the opening such that the lid prevents physical access to the interior chamber and maintains a seal between the lid and the rigid bin; placing one or more items in an interior portion of a second container; (4) placing the second container within the rigid bin such that the first interior is in gaseous communication with the second interior of the second container; (5) using the fastening mechanism to maintain the selectively closeable opening in the closed position; and (6) turning on the ozone generator to cause at least some ozone gas to expel into the first interior portion through the at least one conduit.

### BRIEF DESCRIPTION OF THE DRAWINGS

Various embodiments of an assembly for removing odors from clothing or other items are described below. In the course of this description, reference will be made to the accompanying drawings, which are not necessarily drawn to scale and wherein:

FIG. 1 is an end perspective view of an embodiment of an assembly for removing odors, in a closed orientation;

FIG. 2 is a side perspective view of the assembly of FIG. 1, in an open orientation;

FIG. 3 is a partial end perspective view of the assembly of FIG. 1 showing an ozone generator located inside an exterior pocket of the assembly;

FIG. 4 is an end perspective view of the assembly of FIG. 1, showing an ozone generator located outside of the assembly with a conduit for ozone attached to the ozone generator;

FIG. 5 is a front perspective view of the ozone generator;

FIG. 6 is a partial perspective view of an inside surface of the assembly of FIG. 1; and

FIG. 7 is a front view of an ozone destruction catalyst for use within the assembly of FIG. 1.

FIG. 8 is a perspective view of an assembly for removing odors according to a particular embodiment.

FIG. 9 is a perspective view of the assembly for removing odors of FIG. 8 with a first bag within a second, odor-removing bag.

FIG. 10 is a perspective view of an odor removing assembly according to another embodiment.

FIG. 11 is a perspective view of the odor removing assembly of FIG. 10 with the lid open.

FIG. 12 depicts a second bag being placed within an odor-removing rigid bin as part of an odor removing assembly.

FIG. 13 depicts the second bag of FIG. 12 within the rigid bin of FIG. 12.

### DETAILED DESCRIPTION

Various embodiments will now be described more fully hereinafter with reference to the accompanying drawings. It should be understood that the invention may be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

### Structure of Odor Removal Assembly

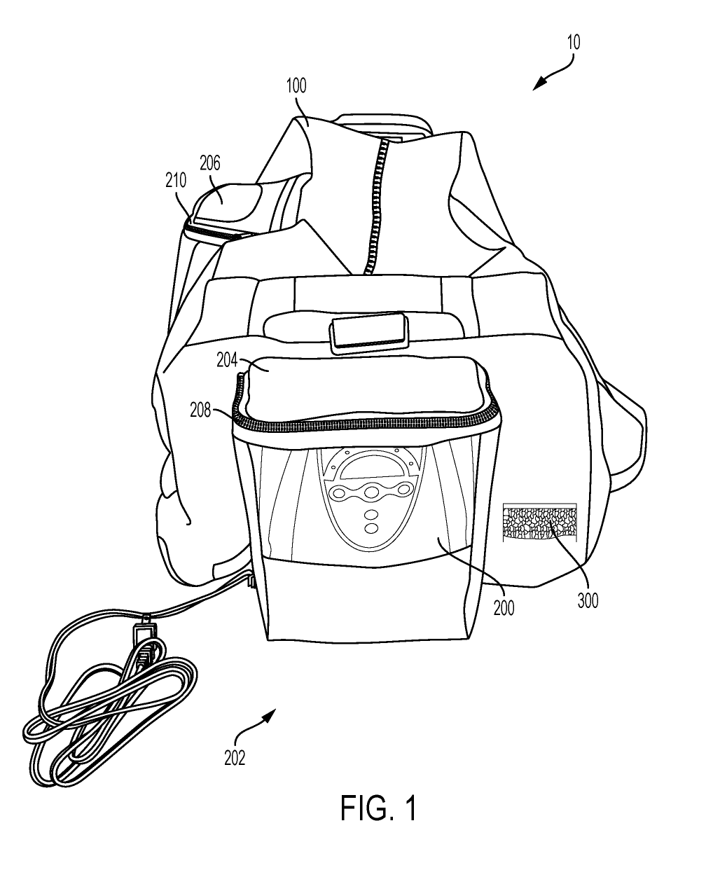

As shown in FIG. 1, an odor removal assembly 10, according to various embodiments, includes: (1) a flexible bag 100; (2) an ozone generator 200; and (3) an ozone destruction catalyst 300. These various components are discussed in more detail below.

Flexible Bag

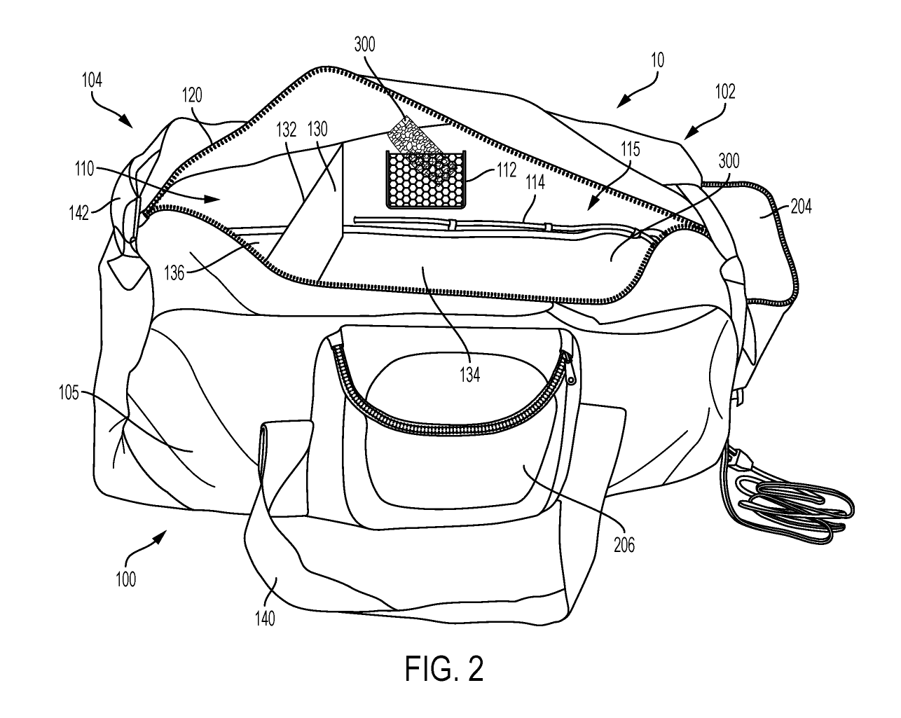

Referring to FIG. 2, the odor removal assembly 10, in various embodiments, includes a flexible bag 100 (or other flexible or non-flexible container) that is configured for storing clothing and/or other items within its interior. In various embodiments, the flexible bag 100 may be a flexible container such as a portable closet. For example, the portable closet may have a removable clothing rod for hanging athletic apparel and may have foldable shelving for holding other athletic gear. In other embodiments, the flexible bag 100 may be a non-flexible container such as a steel closet. In various embodiments, the steel closet may be any suitable size (e.g., 2-10 feet tall, 2-10 feet wide, 2-10 feet deep, etc.). The flexible bag 100 may be any suitable size or shape (e.g., cubical, cylindrical, spherical, conical, hexagonal, etc.). In some embodiments, the flexible bag 100 may include ribbing, piping, or any other suitable structural material to maintain the shape of the flexible bag 100. The flexible bag 100 may be made of fabric or any other suitable flexible material (e.g., fabric, ballistic nylon, plastic, etc.). In various embodiments, the flexible bag 100 may be made of non-flexible material. In particular embodiments, the flexible bag 100 may be made of water-resistant material (e.g., GORE-TEX®). In other embodiments, the flexible bag 100 may be made at least partially from an activated carbon material.

In the embodiment shown in FIGS. 1-7, the flexible bag 100 includes a central housing 105 that defines an interior portion 110. The central housing 105 defines a first end 102 and a second end 104 and an opening 115 intermediate the housing's first and second ends 102, 104 through which a user may selectively access the interior portion 110 of the central housing. In particular embodiments, the opening 115 may be in any suitable shape. In various embodiments, the opening 115 may be parallel to a central axis that extends between the first and second ends 102, 104 of the central housing 105. In other embodiments, the opening 115 may be substantially perpendicular to the central axis that extends between the first and second ends 102, 104 of the central housing 105. In still other embodiments, the opening 115 may be substantially U-shaped.

In various embodiments, when the flexible bag opening 115 is in an open orientation, as shown in FIG. 2, a user may place clothing, or one or more other objects, into the interior portion 110 of the central housing 105 through the opening 115. In particular embodiments, when the opening 115 is in a closed orientation, as shown in FIG. 1, a user is prevented from placing clothing, or other objects, into the interior portion 110 of the central housing 105 though the opening 115.

As shown in FIGS. 1 and 2, the flexible bag 100 may include a fastening mechanism 120 for selectively maintaining the bag opening 115 in a closed orientation. The fastening mechanism 120 may comprise any suitable fastening mechanism (e.g., a zipper; one or more buttons; a hinged door; a hook and loop fastening assembly; a ziplock arrangement, etc.). In particular embodiments, the fastening mechanism 120 may comprise a zipper. In various embodiments, the opening 115 and the fastening mechanism 120 are the same length. For example, where the opening 115 is parallel to the axis that extends between the first and second ends 102, 104 of the central housing 105, the fastening mechanism 120 is also parallel to this axis. In some embodiments, the fastening mechanism 120 may extend the entire length of the central housing 105 from the housing's first end 102 to the housing's second end 104. In other embodiments, the fastening mechanism 120 may cover only a portion of the length intermediate the housing's first and second ends 102, 104. In various embodiments, the fastening mechanism 120 may also include a locking mechanism (not shown). The locking mechanism may be any suitable locking mechanism (e.g., a padlock, a combination lock, etc.). The locking mechanism may be used to prevent unwanted access by children and/or others into the interior portion 110 of the bag 100 by maintaining the fastening mechanism 120 in the closed orientation.

Referring again to FIG. 2, in various embodiments, the interior portion 110 of the central housing 105 may be dimensioned to hold athletic apparel, sporting goods, shoes, and/or any other gear and/or apparel. In some embodiments, the interior portion 110 of the central housing 105 may be coated (e.g., partially or entirely) with an ozone destruction material. In other embodiments, the interior portion 110 of the central housing 105 may include one or more interior pockets 112. In various embodiments, the one or more interior pockets 112 may be made of any suitable flexible material, such as a mesh material.

In some embodiments, the one or more interior pockets 112 may be integrally formed with an interior surface of the central housing. In other embodiments, the one or more interior pockets 112 may be selectively detachable from the central housing's interior surface. In certain embodiments, the one or more interior pockets 112 may include a fastening mechanism (not shown) (e.g., a zipper, one or more buttons, a hook and loop fastening assembly, etc.) for selectively maintaining an opening of one or more of the interior pockets 112 in a closed position to retain items within the interior pockets 112.

In particular embodiments, the one or more interior pockets 112 are configured to hold ozone destruction material such as the ozone destruction catalyst 300. In various embodiments, the one or more interior pockets 112 serve as an ozone destruction catalyst support assembly that is adapted for supporting the ozone destruction catalyst 300 within the interior portion 110 of the flexible bag's central housing 105. In particular embodiments, the ozone destruction catalyst support assembly comprises at least one mesh pocket.

In various embodiments, a partition 130 may be located within the central housing's interior portion 110. The partition 130 may be made of any suitable (e.g., sturdy) material (e.g., foam, cardboard, plastic, etc.). In various embodiments, the partition 130 is made of foam and covered with the same material that the interior of the flexible bag 100 is made of. In some embodiments, the partition 130 may be selectively detachable from the central housing's interior portion 110. For example, an outer circumferential edge 132 of the partition 130 may include hook fastening material that is attachable to loop fastening material running the length of the interior portion 110 of the central housing 105 from the central housing's first end 102 to the central housing's second end 104. In certain embodiments, the size and shape of the partition 130 may correspond to the size and shape of a cross section of the central housing's interior portion 110 such that, when the partition 130 is inserted into the interior portion 110, the partition 130: (1) engages one or more interior walls of the flexible bag 100 and is held in place by friction; and (2) divides the interior portion 110 of the central housing 105 into two separate interior chambers 134, 136.

In particular embodiments, the partition 130 may be adapted to be moved between: (1) a first position, in which the partition 130 cooperates with one or more interior walls of the bag's central housing 105 to form a first interior chamber 134 of a first size; and (2) a second position, in which the partition 130 cooperates with the one or more interior walls of the flexible bag 100 to form a first interior chamber 134 (on the same side of the partition 130) that is of a second size that is different from the first size. In some embodiments, the second size of the first interior chamber 134 is larger than the first size of the first interior chamber 134. In particular embodiments, when: (1) the partition 130 is in the first position, a conduit 114 extends in gaseous communication between an outlet of the ozone generator 222 and the first interior chamber 134; and (2) the partition 130 is in the second position, the conduit 114 extends in gaseous communication between an outlet of the ozone generator 222 and the second interior chamber 136.

Returning to FIG. 1, an exterior portion 202 of the flexible bag 100 may include one or more exterior pockets 204, 206. In various embodiments, the one or more exterior pockets 204, 206 may be made of any suitable material (e.g., flexible material). In particular embodiments, the one or more exterior pockets 204, 206 may be made of mesh material. In other embodiments, the one or more exterior pockets 204, 206 may be made of transparent material such as plastic. In some embodiments, the one or more exterior pockets 204, 206 may be integrally formed with the flexible bag's exterior portion 202. In other embodiments, the one or more exterior pockets 204, 206 may be detachable from the flexible bag exterior portion 202. In yet other embodiments, the one or more exterior pockets 204, 206 may include a fastening mechanism 208, 210 (e.g., zipper, one or more buttons, hook and loop fastening assembly, etc.) for keeping contents within the one or more exterior pockets 204, 206. In particular embodiments, a particular exterior pocket 204 is configured to hold the ozone generator 200. In various embodiments, the exterior pocket 204 is an ozone generator support assembly that is adapted for supporting the ozone generator 200 within the interior portion of the exterior pocket 204.

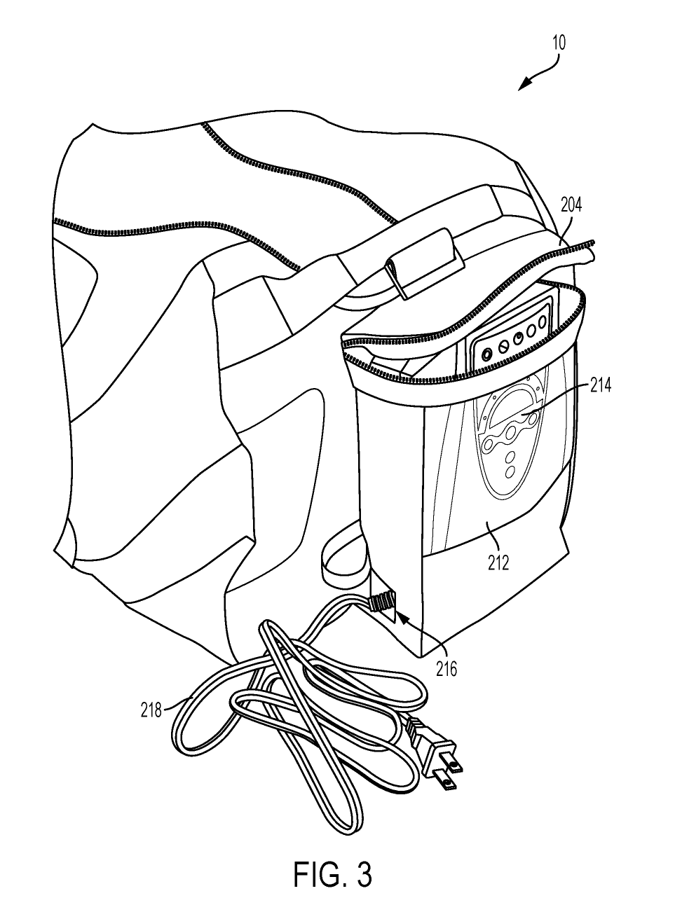

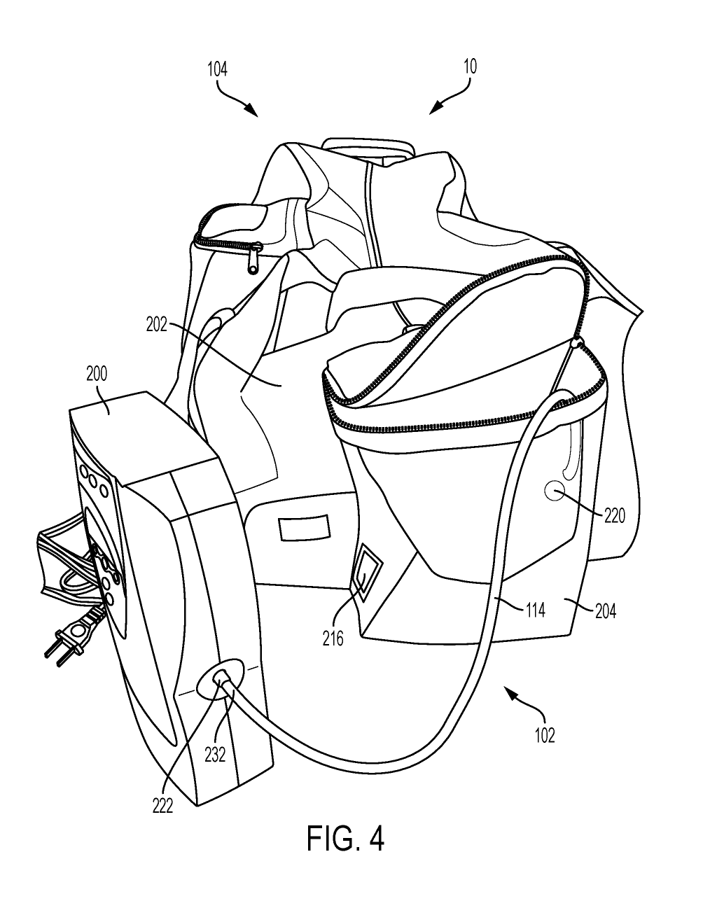

Referring to FIGS. 3 and 4, the exterior pocket 204 is shown in an open orientation. In various embodiments, the exterior pocket 204 has a transparent front portion 212 for viewing the contents of the pocket. For example, when the ozone generator is located within the interior of the exterior pocket 204, a user may view a user interface 214 of the ozone generator 200. In particular embodiments, the exterior pocket 204 has a first opening 216 for allowing a power cord 218 associated with the ozone generator 200 to pass through to the outside of the flexible bag 100. Referring specifically to FIG. 4, in various embodiments, the exterior pocket 204 has a second opening 220 for allowing the conduit 114 to pass through from the ozone generator 200 to the interior portion 110 of the bag 100 so that the conduit 114 extends in gaseous communication between the outlet 222 of the ozone generator 200 to the interior portion 110 of the bag 100.

Referring again to FIG. 2, the flexible bag 100 has one or more straps 140 and one or more handles 142 connected to the flexible bag's exterior portion 202 for carrying and handling of the flexible bag 100. The one or more straps 140 and the one or more handles 142 may be made of any sturdy, flexible material (e.g., woven nylon). In various embodiments, the one or more straps 140 and the one or more handles 142 may be integrally formed with the flexible bag 100. In other embodiments, the one or more straps 140 and the one or more handles 142 may be connected to the exterior portion of the bag 202 in any suitable way. For example, the one or more straps 140 and the one or more handles 142 may be sewn into the exterior portion of the bag 202.

Ozone Generator

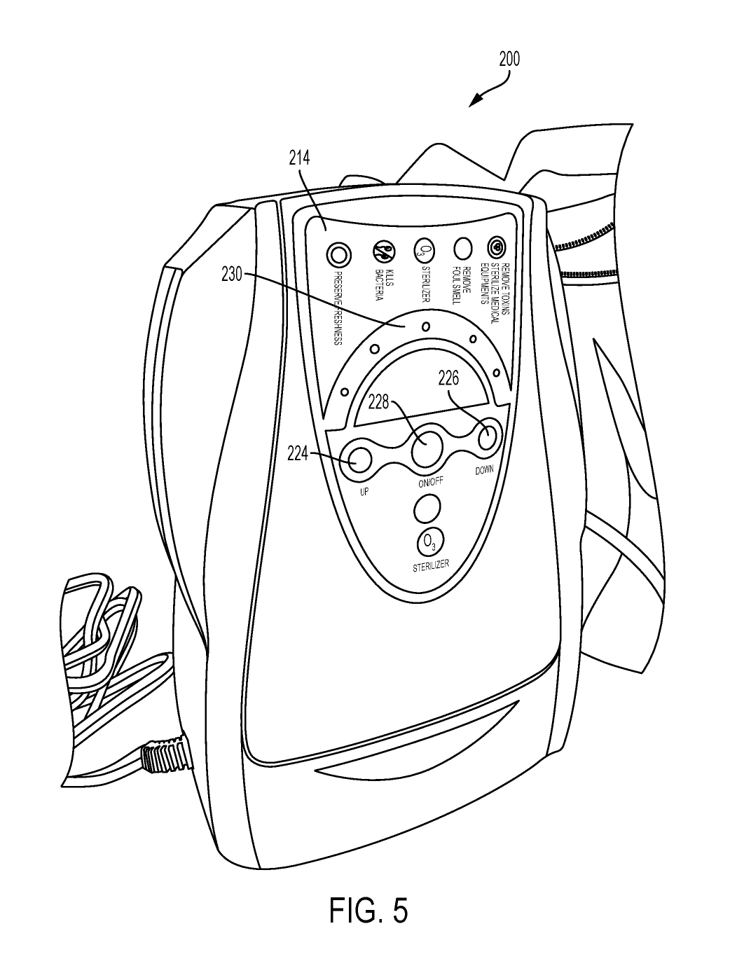

Referring to FIGS. 4 and 5, in various embodiments, the odor removal assembly 10 includes any suitable ozone generator 200 (e.g., an electric ozone generation device) configured to generate ozone. In various embodiments, the ozone generator 200 may be positioned in any suitable location adjacent the flexible bag 100. In particular embodiments, the ozone generator 200 may be disposed adjacent the exterior portion of the bag 100. In other embodiments, the ozone generator 200 may be positioned within one of the one or more exterior pockets 204, 206. In some embodiments, the ozone generator 200 may be positioned so that it is spaced apart from the flexible bag 100. In particular embodiments, the ozone generator 200 may be operatively coupled to the flexible bag 100.

In various embodiments, the ozone generator 200 generates ozone and expels it through the outlet 222, as shown in FIG. 4. The ozone generator 200 may, for example, generate ozone in a concentration that is suitable for removing odors from clothing and other items. In particular embodiments, the ozone generator 200 includes power cord 218 operatively coupled to the ozone generator 200. In some embodiments, the power cord 218 comprises a standard wall plug. In other embodiments, the power cord 218 comprises a 12-volt car plug, or other suitable plug.

In various embodiments, the ozone generator 200 includes a user interface 214 and a computer processor (not shown) for allowing a user to regulate the amount of ozone that the ozone generator 200 generates in a particular deodorizing session. In particular embodiments, the user interface 214 may include control buttons 224, 226 for adjusting the level of ozone expelled from the ozone generator 200 up or down, respectively. In various embodiments, the user interface 214 may also include a power button 228 for turning the ozone generator on or off.

In particular embodiments, the ozone generator 200 may include a timer 230 with a display on the user interface 214 for allowing a user to select the amount of time that the ozone generator will operate in a particular session. In various embodiments, the timer 230 may allow a user to select a specific period of time (e.g., seconds, minutes, hours, etc.). In other embodiments, the timer 230 may allow a user to select a pre-set time limit (e.g., five minutes, 10 minutes, 15 minutes, 30 minutes, one hour, etc.). In some embodiments, the timer 230 may use a suitable selectable program for setting the timer (e.g., “preserve freshness”, “kill bacteria”, “sterilize”, “remove foul smell”, and “remove toxins/sterilize medical equipment”). For example, in a particular embodiment, the ozone generator may be adapted to run for five minutes in response to a user selecting a “preserving freshness” button. As a further example, for items that require a significant amount of deodorizing, a user may select a program that is longer in duration.

In various embodiments, the ozone generator 200 may include, or otherwise be coupled to, an ozone sensor (not shown), which may be adapted to communicate with a controller within the ozone sensor via a suitable communications channel (e.g., a suitable wired or wireless communications channel). The ozone sensor may be adapted for sensing the concentration of ozone adjacent the sensor. In particular embodiments, the controller may be adapted to prevent the operation of the ozone generator (e.g., turn off the ozone generator, or prevent the ozone generator from being turned on) at least partially in response to (e.g., in response to) the ozone sensor sensing one or more predetermined ozone levels (e.g., ozone concentration levels) adjacent the ozone sensor.

In some embodiments, the ozone sensor may be positioned at any suitable location [e.g., inside the flexible bag 100 (e.g., within the interior portion 110 of the central housing 105), or outside of the flexible bag 100 (e.g., adjacent the ozone generator 200)]. As noted above, in various embodiments, the ozone generator's controller may be adapted to turn off or otherwise modify the ozone generator's output of ozone in response to the ozone concentration measured by the ozone sensor reading an ozone concentration level above a pre-determined threshold, and thereby satisfying a pre-determined condition. For example, the ozone generator 200 may be adapted to turn off for safety reasons and/or to prevent potential damage to clothing or other items within the flexible bag 100 caused by over-exposure to excessively high levels of ozone. In some embodiments, the controller may turn off the ozone generator 200 in response to the ozone sensor sensing an ozone level above a particular pre-determined threshold outside of the flexible bag 100. In other embodiments, the controller may turn off the ozone generator 200 in response to the ozone sensor sensing an ozone level above a particular pre-determined threshold outside of the flexible bag 100 within an interior portion of the flexible bag 100 (e.g., within the central housing's interior 110).

In particular embodiments, the ozone generator's controller may be adapted to turn off the ozone generator 200 at least partially in response to (e.g., in response to) the ozone sensor and/or the controller sensing that the flexible bag 100 (e.g., the central housing 105) has been opened (or is otherwise in an open position). In particular embodiments, the controller (which may be computerized) may, for example, be programmed to assume that the central housing 105 has been opened in response to detecting a relatively sudden drop in ozone concentration within the bag (e.g., a concentration drop above a predetermined threshold within a predetermined period of time).

Similarly, the controller may be programmed to assume that the central housing 105 is open based, at least in part, on: (1) the length of time that the ozone generator has been running; and (2) the change in ozone concentration within the interior of the bag (e.g., central housing 105) over time. For example, the controller may be programmed to assume that the bag (e.g., the bag's central housing 105) is in an open orientation if the ozone concentration level within the bag's interior (e.g., within the interior portion 110 of the central housing 105) doesn't reach a pre-determined threshold level in response to the ozone generator running for a pre-determined period of time.

In other embodiments, the ozone generator 200 may include any other suitable type of sensor for sensing whether the bag 100 is in an open or closed orientation (e.g., a suitable mechanical sensor or other sensor). In such embodiments, the controller may be adapted to prevent operation of the ozone generator in response to this sensor sensing that the bag 100 is in an open orientation.

Conduit for Transferring Ozone from the Ozone Generator to the Interior of the Flexible Bag

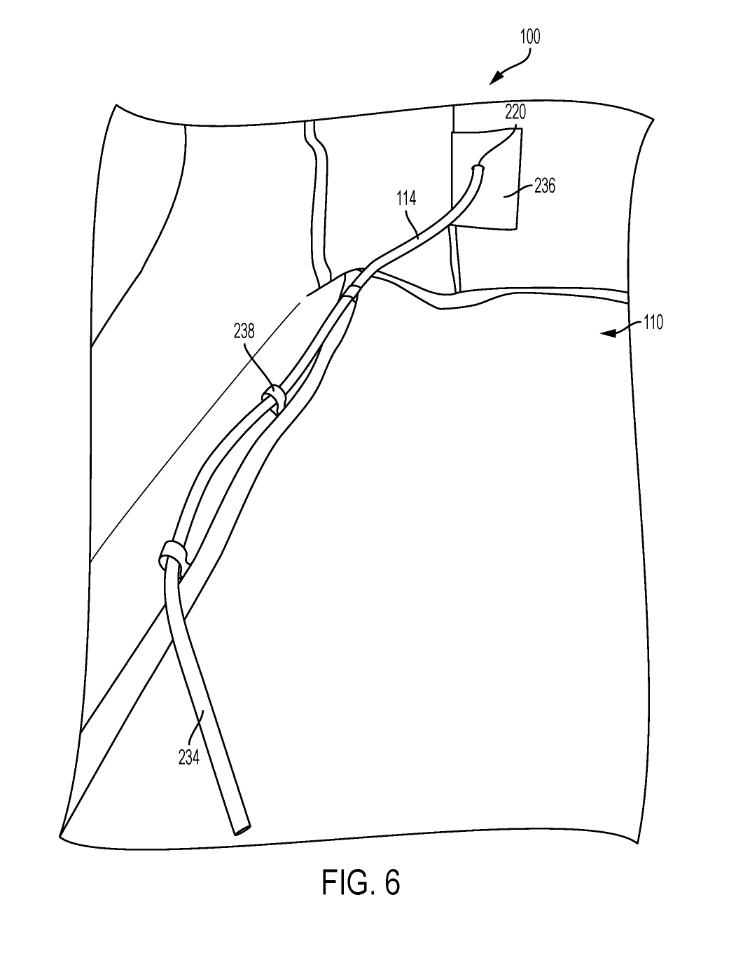

Referring to FIGS. 4 and 6, in various embodiments, the ozone removal assembly 10 includes a conduit 114 that is disposed at least partially within the interior portion of the bag 100. The conduit 114 is in gaseous communication between the outlet of the ozone generator 222 and the interior portion of the flexible bag 100 for transferring ozone from the ozone generator 200 to the interior portion of the bag 100. In particular embodiments, the conduit 114 is adapted to facilitate the flow of ozone from the ozone generator outlet 222 into the interior portion of the flexible bag 100. Referring specifically to FIG. 4, in particular embodiments, the conduit 114 has a first end 232 operatively coupled to the ozone generator 200. Referring specifically to FIG. 6, according to various embodiments, the conduit 114 has a second end 234 disposed within the interior portion of the bag 100. In various embodiments, the conduit second end 234 may be positioned in any suitable location within the interior portion of the bag 100. For example, the conduit second end 234 may be in a central portion of the interior portion of the bag (e.g., substantially midway between the flexible bag's first and second ends 102, 104). In some embodiments, the conduit 114 may extend through the pocket second opening 220 such that the conduit 114 extends between the exterior pocket 204 and the interior portion of the bag 100. In particular embodiments, the second opening 220 may be surrounded by backing 236 to preserve the size and shape of the second opening 220.

In various embodiments, the conduit 114 may be tubing or any other suitable conduit. In particular embodiments, the conduit is made of flexible material. In other embodiments, the conduit is made of non-flexible material. In various embodiments, the conduit 114 may be elongated and relatively long (e.g., 1-15 feet in length). In some embodiments, the conduit 114 may be integrally formed with the interior portion of the bag 100. In other embodiments, the interior portion of the bag 100 may include one or more straps 238 or other fastening arrangements for holding the conduit 114 in place. The one or more straps 238 may be made from any suitable sturdy material (e.g., woven nylon, etc.). In various embodiments, the one or more straps 238 may be connected to the interior portion of the bag 100 in any suitable way. For example, the one or more straps 238 may be sewn into the interior portion of the bag 100.

Ozone Destruction Catalyst

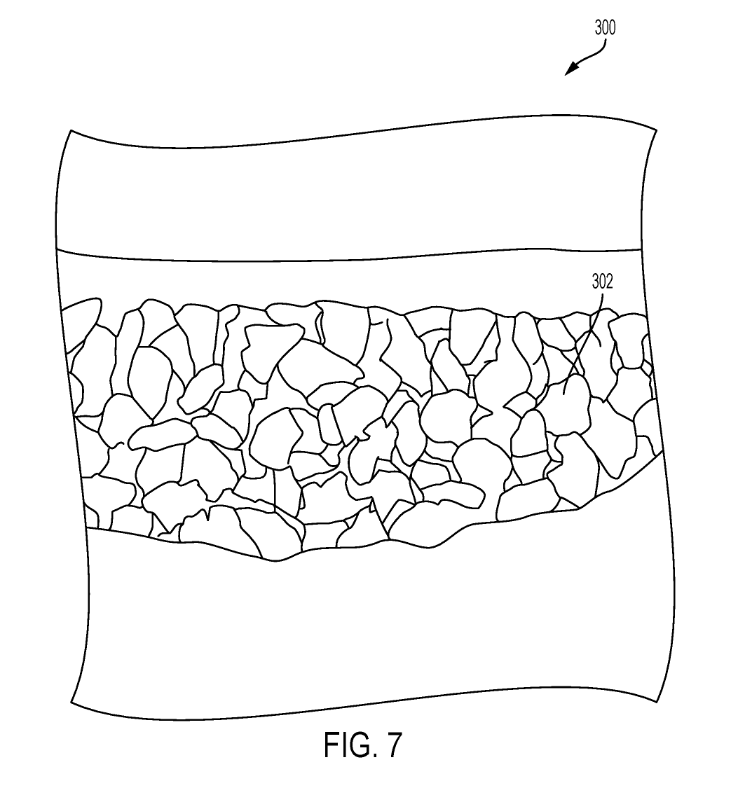

Referring to FIG. 7, the ozone removal assembly 10, according to various embodiments, includes an ozone destruction catalyst. In particular embodiments, the ozone destruction catalyst 300 may comprise any suitable catalyst for destroying ozone (e.g., CuO and MnO₂, Au, Pd, Pt, etc.). The ozone destruction catalyst 300 may, in various embodiments, catalyze ozone (O₃) into oxygen (O₂) through a catalytic reaction. The ozone destruction catalyst 300 may be used, for example, to prevent damage to clothing or other items within the bag and/or to prevent over-exposure to ozone by humans and other living organisms outside of the bag 100 caused by excessively high levels of ozone.

As previously discussed, the one or more interior pockets 112 may be configured to hold the ozone destruction catalyst 300 such that the ozone destruction catalyst 300 is disposed and maintained within the interior portion of the flexible bag 100. In various embodiments, the ozone destruction catalyst 300 may be in any suitable form (e.g., in the form of one or more spheres, cylinders, honeycombs etc.). In some embodiments, the ozone destruction catalyst 300 is a pellet catalyst. In particular embodiments, the ozone destruction catalyst 300 and/or the flexible bag 100 are configured so that the ozone destruction catalyst 300 may be selectively removed from the interior portion 110 of the flexible bag 100. In such embodiments, if the ozone destruction catalyst 300 needs to be replaced, a user may remove the ozone destruction catalyst 300 (e.g., from the interior pocket 112) and replace it with new ozone destruction catalyst.

### Assembling the Ozone Removal Assembly

Referring generally to FIGS. 1-6, and especially FIG. 4, the ozone removal assembly 10 may be assembled by placing the conduit 114 into the exterior pocket 204 and then through the exterior pocket second opening 220 that provides a gateway into the interior portion 110 of the bag's central housing 105. Once the conduit 114 is positioned so that it extends through the second opening 220, the conduit 114 may then be passed through the one or more straps 238 located in the interior portion of the flexible bag 100. After moving the conduit 114 into place, the ozone generator's power cord 218 may be pulled through the exterior pocket's first opening 216 and the ozone generator 200 may then be placed inside the exterior pocket 204. Once the ozone generator 200 is in place, the fastening mechanism 208 may be fastened to secure the ozone generator 200 within the exterior pocket 204. With the conduit second end 234 positioned midway between the flexible bag's first and second ends 102, 104 a user may position the partition intermediate the conduit second end 234 and the flexible bag's first end 102. The user may then place the ozone destruction catalyst 300 into the mesh interior pocket 112.

### Additional Embodiments of an Ozone Removal Assembly

In various embodiments, an ozone removal assembly may include one or more features that differ from, or are in addition to those discussed above. In particular, some embodiments of an ozone removal assembly may comprise: (1) a first container; (2) an ozone generator; and (3) a second container configured to be placed within (e.g., at least partially within) the first container. In such embodiments, the ozone generator may be operatively coupled to the first container such that ozone generated by the ozone generator is at least partially directed within an interior of the first container. The second container may, for example, then be placed within the interior portion of the first container. In particular embodiments, the first container may comprise a flexible bag, a substantially rigid (e.g., rigid) bin, or any other suitable container. In some embodiments, the first container comprises an odor-removing container that is configured to remove the odors of items placed in the second container when the second container is put in the first container. Various exemplary embodiments of such a container-within a container arrangement are described below.

Flexible Bag within a Flexible Bag Arrangement

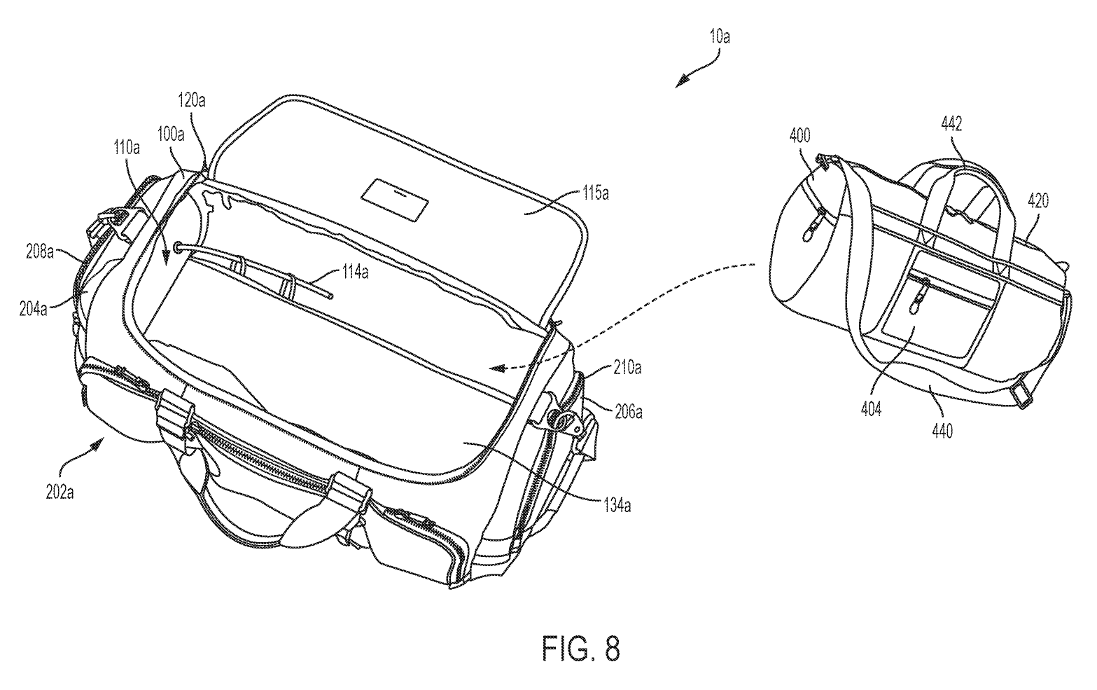

As shown in FIG. 8, an odor removal assembly 10a, according to a particular embodiment, includes: (1) a first flexible bag 100a; (2) an ozone generator (not pictured) disposed within a pocket 204a of the first flexible bag 100a; and (3) a second flexible bag 400. These various components are discussed in more detail below.

In various embodiments, the odor removal assembly 10a includes a first flexible bag 100a (or other flexible or non-flexible container) that is configured for storing a second container (e.g., a second flexible bag) within its interior. In various embodiments, the first flexible bag 100a may include any suitable bag such as, for example, any suitable duffel bag or carrying case. The first flexible bag 100a may be any suitable size or shape (e.g., cubical, cylindrical, spherical, conical, hexagonal, etc.). In some embodiments, the first flexible bag 100a may include ribbing, piping, or any other suitable structural material to maintain the shape of the first flexible bag 100a (e.g., in order to substantially maintain a shape of the first flexible bag 100a when the first flexible bag 100a is empty).

The first flexible bag 100a may be made of fabric or any other suitable flexible material (e.g., fabric, ballistic nylon, plastic, etc.). In various embodiments, the first flexible bag 100a may be made of non-flexible material. In particular embodiments, the first flexible bag 100a may be made of water-resistant material (e.g., GORE-TEX®). In other embodiments, the first flexible bag 100a may be made at least partially from an activated carbon material.

In various embodiments, the first flexible bag 100a may comprise any suitable material that is at least partially impervious to ozone. For example, in such embodiments, the first flexible bag 100a may comprise a material that limits ozone leakage from within the first flexible bag 100a to an environment in which the first flexible bag 100a is located such that an ozone concentration in the surrounding environment remains below about 10 parts per million. In other embodiments, the first flexible bag 100a may comprise a material that is sufficiently impervious to ozone to limit ozone leakage (e.g., seepage) from the first flexible bag 100a to the environment in which the first flexible bag 100a is located such that the ozone concentration in the surrounding environment remains, for example: (1) less than about 5 ppm; (2) less than 0.2 ppm; (3) less than 0.1 ppm; (4) less than 0.08 ppm; (5) less than 0.05 ppm; and/or (6) any other level that may pose a health risk to people when exposed to such an ozone concentration. In various embodiments, the first flexible bag 100a may comprise any suitable carbon nanomaterial.

In various embodiments, first flexible bag 100a may comprise a material that is sufficiently impervious to ozone to limit ozone leakage (e.g., seepage) from the first flexible bag 100a to the environment in which the first flexible bag 100a is located such that the ozone concentration in the surrounding environment is less than a particular Occupational Safety and Health Administration guideline related to ozone exposure. For example, the ozone imperviousness of the material that the first flexible bag 100a comprises may be based on a type of activity performed by humans in the environment in which the bag is located (e.g., or will be generally utilized or located). For example, a flexible bag 100a meant to be kept and used in a confined area such as a vehicle or garage may comprise a material that is substantially impervious to ozone to substantially prevent ozone from leaking into the car or garage while the odor removal assembly is in use. As may be understood by one skilled in the art, having a first flexible bag 100a that is at least partially impervious to ozone leakage may be beneficial to prevent ozone leakage from within the bag to a surrounding environment (e.g., into a car, garage, home, etc.), which may pose health risks to users and others. In some embodiments, the ozone generator comprises one or more ozone sensors configured to measure an ozone concentration in an area around the ozone generator (e.g., an area around the first container).

In the embodiment shown in FIG. 8, the first flexible bag 100 defines an interior portion 110a selectively accessible via an opening 115a. In various embodiments, the opening 115a may be in any suitable shape. In the embodiment shown in FIG. 8, the opening 115a is substantially U-shaped (e.g., U-shaped).

In various embodiments, when the flexible bag opening 115a is in an open orientation, as shown in FIG. 8, a user may place any suitable object (e.g., a second flexible bag), into the interior portion 110a of the first flexible bag 10a through the opening 115. In particular embodiments, when the opening 115a is in a closed orientation, a user may be prevented from placing objects, into the interior portion 110a.

As shown in FIG. 8, the first flexible bag 100a may include a fastening mechanism 120a for selectively maintaining the bag opening 115a in a closed orientation. The fastening mechanism 120 may comprise any suitable fastening mechanism (e.g., a zipper; one or more buttons; a hinged door; a hook and loop fastening assembly; a ziplock arrangement, etc.). In the embodiment shown in this figure, the fastening mechanism 120 comprises a zipper. In various embodiments, the fastening mechanism 120a may extend along substantially all of an edge of the opening 115a such that when the fastening mechanism 120a is maintaining the opening 115a in a closed position, the fastening mechanism 120a forms a seal that at least partially prevents ozone from leaking out of the opening 115a.

In various embodiments, for example, the fastening mechanism 120a comprises an airtight zipper. In such embodiments, the zipper may comprise a toothed zipper (e.g., having 2 rows of zipper teeth) and waterproof sheeting that is bonded to the first flexible bag 100a and wrapped around an outer portion of each row of zipper teeth. In particular embodiments, the waterproof sheeting comprises a suitable fabric-reinforced polyethylene material or other suitable material. In particular embodiments, when the locking mechanism 120a is in the closed position (e.g., locked position), the two sheets of waterproof sheeting on the opposing zipper teeth are configured to squeeze against one another to form a substantially air-tight (e.g., air-tight) seal along the locking mechanism 120a (e.g., zipper). In such embodiments, an air-tight locking mechanism 120a may, for example, at least partially prevent ozone from leaking out of the first flexible bag 100a to a surrounding environment in which the first flexible bag 100a is located. The air-tight nature of the locking mechanism 120a may, in various environments, be beneficial in avoiding potential issues related to ozone exposure by people in the vicinity of the ozone removal assembly 10a. For example, in an embodiment in which the ozone removal assembly is in a user's vehicle, it may be desirable for the first flexible bag 100a to not leak ozone into the vehicle (e.g., due to any potential health risks due to ozone exposure to people in the vehicle).

In various embodiments, the first flexible bag's interior portion 110a may define an interior chamber 134a of any suitable size or dimension (e.g., rectangular, etc.). In various embodiments, the interior chamber 134a may be sufficiently large to accommodate a second flexible bag 400 (e.g., or inflexible bag) within the interior chamber 134a. In further embodiments, as discussed above, the odor removal assemble 10a includes a conduit 114 that is disposed at least partially within the interior chamber 134a of the first flexible bag 100a. The conduit 114 is in gaseous communication between the outlet of the ozone generator (not pictured) and the interior chamber 134a of the flexible bag 100a for transferring ozone from the ozone generator to the bag's interior chamber 134a.

In various embodiments, an exterior portion 202a of the first flexible bag 100a may include one or more exterior pockets 204a, 206a. In various embodiments, the one or more exterior pockets 204a, 206a may be made of any suitable material (e.g., flexible material). In particular embodiments, the one or more exterior pockets 204a, 206a may be made of mesh material. In other embodiments, the one or more exterior pockets 204, 206a may be made of transparent material such as plastic. In some embodiments, the one or more exterior pockets 204a, 206a may be integrally formed with the flexible bag's exterior portion 202a. In other embodiments, the one or more exterior pockets 204a, 206a may be detachable from the first flexible bag exterior portion 202a. In yet other embodiments, the one or more exterior pockets 204a, 206a may include a suitable fastening mechanism 208a, 210a (e.g., zipper, one or more buttons, hook and loop fastening assembly, etc.) for keeping contents within the one or more exterior pockets 204a, 206a.

In particular embodiments, a particular exterior pocket 204a is configured to hold the ozone generator 200 described above. In such embodiments the conduit 114a may extend from the particular exterior pocket 204a to the bag's interior chamber 134a via any suitable opening (e.g., a hole, slit, etc. between the particular exterior pocket 204a and the exterior chamber 134a). In various embodiments, the suitable opening between the particular exterior pocket 204a and the exterior chamber 134a is sufficiently airtight when the conduit 114a is inserted through the opening such that air flow is at least partially restricted (e.g., fully restricted) between the particular exterior pocket 204a and the exterior chamber 134a through the opening (e.g., other than via the conduit 114a). In such embodiments, the opening may comprise a suitable gasket (e.g., made of rubber or other material).

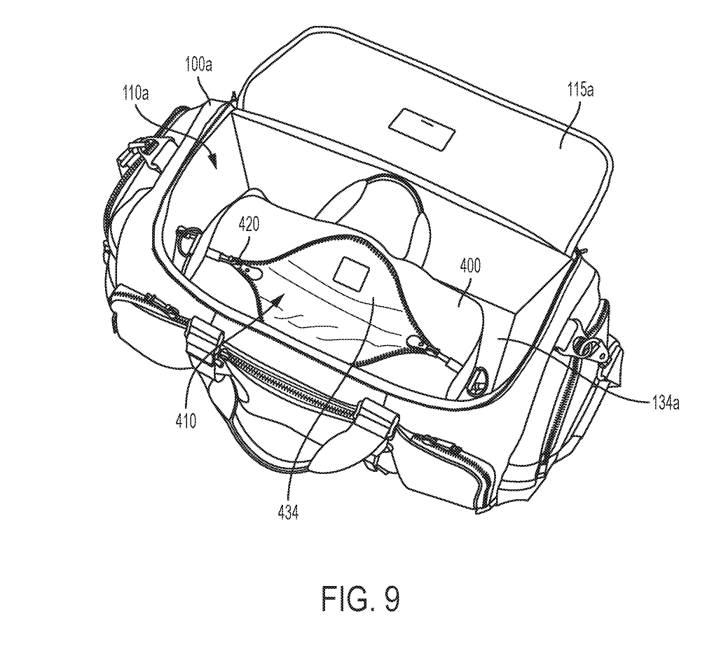

In the embodiment shown in FIG. 8, the odor removal assembly 10a further comprises a second flexible bag 400. The second flexible bag 400 may include any suitable container for holding one or more items (e.g., clothing, shoes, etc.) and, in various embodiments, comprises: (1) one or more straps 440; (2) one or more handles 442; (3); one or more exterior pockets 404 and a locking mechanism 420 for selectively providing access to an interior portion of the second flexible bag 400. As may be understood from FIGS. 8 and 9, the second flexible bag 400 is dimensioned to fit within the first flexible bag 100a (e.g., its interior). As shown in FIG. 9, the second flexible bag 400 defines an interior portion 410 accessible via an opening and further defines an interior chamber 434 configured to hold the one or more items.

In various embodiments, the second flexible bag 400 comprises any suitable material (e.g., e.g., fabric, ballistic nylon, mesh, plastic, etc.). In particular embodiments, the second flexible bag 400 comprises a material that is at least partially permeable to ozone (e.g., such that ozone within the first flexible bag 100a at least partially penetrates through an exterior portion of the second flexible bag 400 into the second flexible bag's interior chamber 434 in order to at least partially deodorize any items within the second flexible bag 400). In other embodiments, the odor removal assembly 10a is configured so that, when the second flexible bag 400 is placed within the first flexible bag 100a in order to at least partially deodorize any items within the second flexible bag 400, the second flexible bag's locking mechanism 420 is in the open position such that the second flexible bag's interior portion 410 is at least partially open to the first flexible bag's interior portion 110a). In various embodiments, this arrangement with the second flexible bag's locking mechanism 420 in the open position may allow a free flow of ozone between first flexible bag's interior portion 110a and the second flexible bag's interior portion 410 (e.g., enables ozone to flow from the first flexible bag's interior portion 110a to the second flexible bag's interior portion 410. In various other embodiments, the first and second flexible bags respective interior portions are in gaseous communication with one another (e.g., in any other suitable way).

Flexible Bag within a Rigid Bin Arrangement

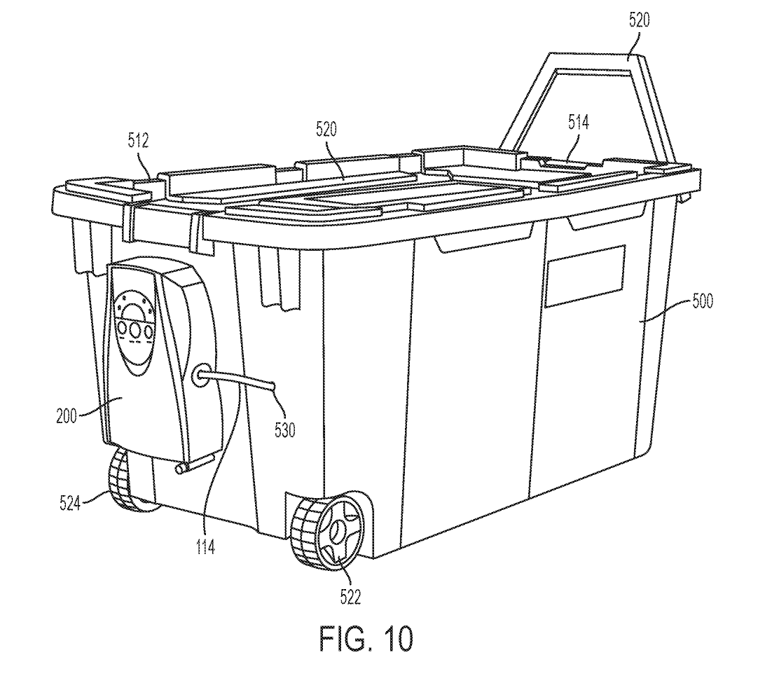

FIGS. 10-13 show an embodiment in which an odor removal assembly comprises: (1) a substantially rigid bin 500; (2) an ozone generator; (3) and a flexible bag 400. This embodiments is similar to the embodiments described above with respect to the flexible bag within a flexible bag arrangement described above. It should be understood by one skilled in the art that other embodiments including other types of containers that serve as an exterior container and/or an interior container that is placed within the exterior container would be suitable for use in the odor removal assembly including the container-within-a-container arrangement described herein. For example, in various embodiments, a first or second container may comprise, for example, a closet (e.g., a free-standing or built-in closet), an entire room, a car trunk, a washing machine, a dryer, a drum, or any other suitable container). Specific embodiments described herein are for illustrative purposes.

FIG. 10 shows a perspective view of a substantially rigid (e.g., rigid) bin 500 that makes up part of an odor removal assembly according to a particular embodiment. In the embodiment shown in this figure, the rigid bin 500 comprises: (1) a substantially rectangular housing 502; (2) two wheels 522, 524 disposed adjacent a lower portion of the housing 502; (3) a handle 520 disposed adjacent an upper portion of the housing 502; (4) a lid 520; (5) and two locking mechanisms 512, 514 for maintaining the lid in a closed position.

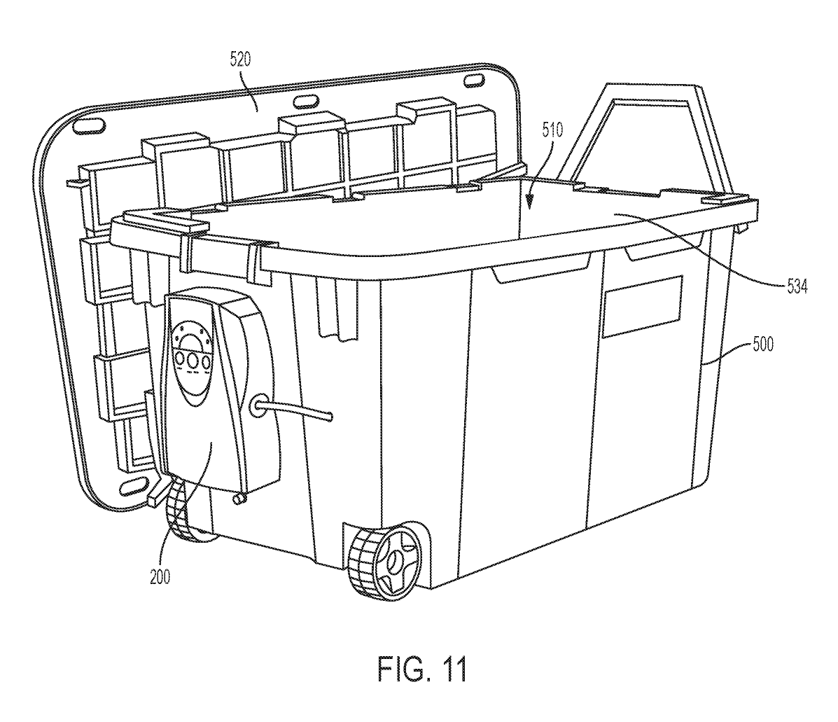

As shown in this figure, the housing 502 defines an opening 530 through which the ozone generator's conduit 114 extends into the rigid bin's interior. FIG. 11 shown the rigid bin's interior 510 which defines an interior chamber 534 and is accessible via an opening. As may be understood from FIGS. 10 and 11, the locking mechanisms 512, 514 are configured to maintain the lid 520 adjacent the bin's opening in order to selectively prevent and allow access to the interior chamber 534. In various embodiments, when the locking mechanisms 512, 514 are maintaining the lid 520 adjacent the bin's opening, the lid 520 forms a substantially air tight seal with the rigid bin 500 such that air (e.g., ozone) cannot escape the bin's interior chamber 534 via a contact point between the lid 520 and the bin 500. In various embodiments, the seal between the lid 520 and bin 500 may comprise rubber or any other suitable material for maintaining a substantially tight seal between the lid 520 and the bin 500 when the lid is in the closed position.

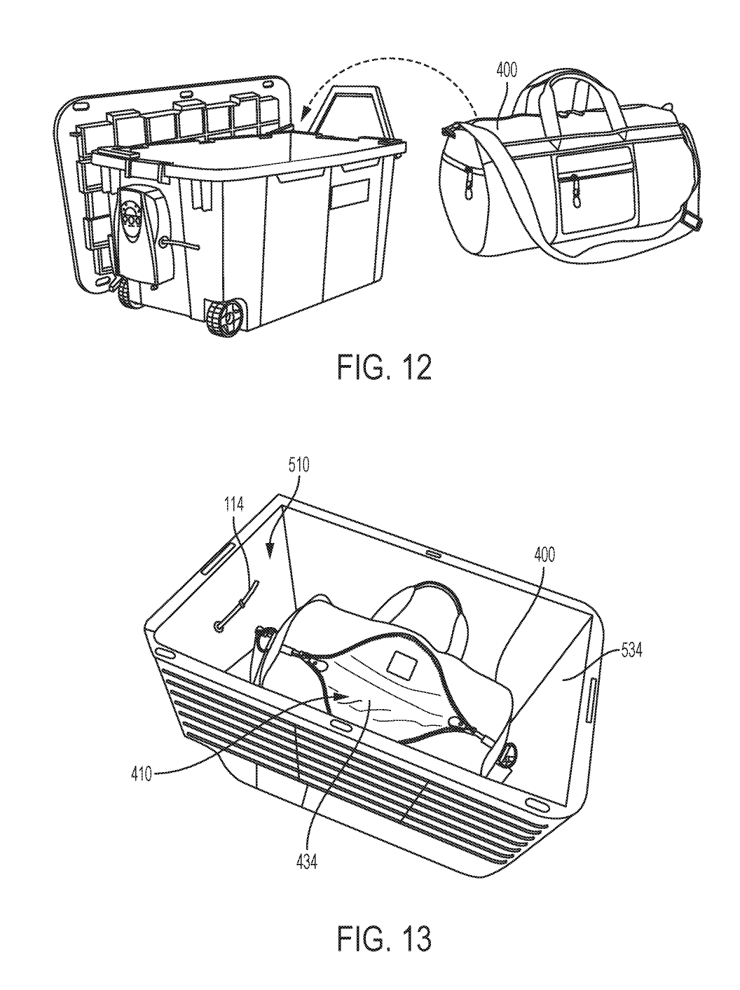

FIGS. 12 and 13 show a second flexible bag 400 being placed within the rigid bin's interior chamber 534. As may be understood from these figures and from the description above with respect to the flexible bag in a flexible bag arrangement with respect to FIGS. 8 and 9, when the second flexible bag 400 is within the interior chamber 534 of the rigid bin 500, ozone generated by the ozone generator may enter the second flexible bag's interior portion 434 (e.g., via an opening in the second flexible bag 400, through an ozone permeable material that makes up at least part of the second flexible bag 400, or in any other suitable manner).

### Exemplary Ozone Generator Power Sources

Various embodiments of an ozone generator may include any suitable power source or power supply. For example, in various embodiments, the ozone generator is configured to utilize any suitable AC or DC power source such as, for example, any suitable battery power of any type (e.g., disposable, rechargeable, etc), any suitable AC power supply (e.g., from a wall outlet), any suitable USB power source, or any other suitable power source. In particular embodiments, the ozone generator is configured to utilize a car battery (e.g., 12 Volt battery) as a power source, for example, via a suitable cigarette lighter adapter. In various embodiments, such as the embodiments shown in FIG. 10, a rigid bin 500 or other carrier may optionally comprise one or more suitable solar chargers comprising one or more solar cells (e.g., photovoltaic cells) for converting light energy into electricity, for example, via the photovoltaic effect. In particular embodiments, for example, the lid may comprise one or more solar cells. In still other embodiments, any other suitable portion of the odor removing assembly may comprise any suitable number or arrangement of solar cells. In various embodiments, the one or more solar cells are configured to charge a rechargeable battery from which the ozone generator draws power.

### Use of the Ozone Removal Assembly

In various embodiments, after the ozone removal assembly 10 has been assembled it is ready for use. In using the ozone removal assembly 10, a user may place one or more items such as an athletic jersey, athletic shoes, hunting gear, and/or other sporting gear or other items into the interior of the bag's central housing 105 through the flexible bag's opening 115. These items may be items that the user wishes to deodorize. In various embodiments, the user may place items with similar levels of odor into the flexible bag 100 at the same time. For instance, if the user has socks and shoes from a short walk as well as hunting apparel from a long weekend hunt that needs to be deodorized, the user may desire to deodorize these items separately as the hunting gear may require a longer deodorizing session to be fully deodorized. Once the items have been placed into the flexible bag 100, the user may close the flexible bag's opening 115 by moving the fastening mechanism 120 from the open orientation to the closed orientation.

In some embodiments, the user may then lock the fastening mechanism 120 using a suitable locking mechanism as described above. When the fastening mechanism 120 is in the locked orientation, the user may plug the ozone generator′ power cord 218 into a suitable power source (e.g., a wall plug or a car plug) and then turn the ozone generator 200 on using the power button 228. A user may select the level of ozone to be expelled from the ozone generator 200 by pressing either the up button 224 or the down button 226 depending upon the length of the time and level of ozone required to deodorize the items placed into the flexible bag 100. For example, for particularly foul odors, the user may elect to have the ozone generator 200 generate ozone for 30 minutes. For mild odors, the user may, for example, elect to have the ozone generator 200 generate ozone for a shorter period of time, such as five minutes.

In using some embodiments of the ozone removal assembly 10, if the assembly's ozone sensor senses that the level of ozone within the bag exceeds a pre-determined threshold, the ozone generator's controller will turn off the ozone generator 200 so that ozone is no longer expelled from the ozone generator 200. Similarly, if the bag opening sensor senses that the bag has been opened, the automatic shutoff system will turn off the ozone generator 200 so that ozone is no longer expelled from the ozone generator 200.

Use of Dual Container Odor Removal Assembly Embodiment

As may be understood from FIGS. 8-9 and 12-13, when using an ozone removal assembly that includes two containers, a user may place one or more items that the user wishes to deodorize into an interior portion 434 of a second flexible bag. The user may then optionally close a locking mechanism 420 (e.g., zipper) of the second flexible bag with the one or more items within it. In such embodiments, the user may then place the second flexible bag 400 into a second container that includes an ozone generator. In various embodiments, the second container may comprise a first flexible bag 100a (e.g., as shown in FIGS. 8 and 9) or a rigid bin 500 (e.g., as shown in FIGS. 12 and 13).

As may be understood from these figures, once the second flexible bag has been placed within the first container, the user may close the first container. In the embodiment that includes a first flexible bag 100a shown in FIGS. 8 and 9, the user may, for example, close the fastening mechanism 120a (e.g., zipper) of the first flexible bag 100a while the second flexible bag 400 is disposed within the first flexible bag's interior portion 134a. As may be understood by one skilled in the art, while the first flexible bag is closed, the first flexible bag is substantially impervious to ozone (e.g., is configured to maintain any ozone directed via the conduit 114 into the first flexible bag's interior portion 134 within the first flexible bag 100a so that ozone does not leak out of the first flexible bag 100a into a surrounding environment). The user may then turn on the ozone generator 200 which generates ozone that flows into the first flexible bag's interior portion 134 via the conduit 114. The ozone then enters the interior portion 434 of the second flexible bag 400 (e.g., via one or more openings or by permeating a material that comprises at least a portion of the second flexible bag 400) and deodorizes the one or more items within.

Similarly, in embodiments that include a rigid bin 500 as shown in FIGS. 12 and 13, the user may, for example, affix the lid 520 to the rigid bin 500 using the one or more fastening mechanisms 512, 514 while the second flexible bag 400 is disposed within the bin's interior portion 534. As may be understood by one skilled in the art, while the bin is closed, the bin is substantially impervious to ozone (e.g., is configured to maintain any ozone directed via the conduit 114 into the bin's interior portion 534 within the rigid bin 500 so that ozone does not leak out of the rigid bin 500 into a surrounding environment). The user may then turn on the ozone generator 200 which generates ozone that flows into the bin's interior portion 534 via the conduit 114. The ozone then enters the interior portion 434 of the second flexible bag 400 (e.g., via one or more openings or by permeating a material that comprises at least a portion of the second flexible bag 400) and deodorizes the one or more items within.

In other embodiments, the odor removal assembly may comprise an ozone generator and a conduit. In such embodiments, the odor removal assembly may further comprise instructions for a user to insert a second end of the conduit in any container that the user has in order to use that container as an odor-removing container. The instructions may, for example, instruct the user to insert the conduit into a storage bin (e.g., a plastic storage bin). The instructions may, for example, instruct the user to cut a hole in an exterior of the storage bin and run the conduit through the whole. In such embodies, the instructions may further instruct the user to seal the hole around the conduit (e.g., using duct tape or other adhesive). In other embodiments, the instructions may direct the user to simply insert the conduit into the opening at the top of the bin and close the lid over the conduit.

### Exemplary Use

Sports Equipment

In a particular example of a user using the ozone removal assembly 10, a user desiring to deodorize sports equipment such as shoes, socks, shorts, and a shirt from a long run may set up the ozone removal assembly 10 in the user's garage by plugging the ozone generator's power cord 218 into a wall outlet located in the user's garage. After placing the sports equipment into the flexible bag 100 and closing the bag 100, the user may consult the ozone generator user interface 214 regarding the correct length of time to set the timer 230 for. Because the user is desiring to kill any bacteria that may be on the sports equipment, the user may turn the ozone generator 200 on and press the up button 224 once to set the ozone generator 200 to a “kill bacteria” setting. This, may, for example, turn the ozone generator 200 on for 10 minutes, which may be a suitable amount of time for killing bacteria on items.

Once the full 10-minute cycle has been completed, the user may leave the sports equipment in the flexible bag 100 with the fastening mechanism 120 (e.g., a zipper) closed to allow the ozone destruction catalyst 300 to catalyze the remaining ozone in the bag 100. The user may then remove the sports equipment from the bag and test the odor of the sports equipment by smelling it. If the sports equipment requires further deodorizing, the user may place the sports equipment back into the bag 100 and repeat the steps listed above. If the user determines that undesired odor is still prevalent in the sports equipment, the user may, for example, increase the level of ozone produced by the ozone generator 200 from 10 minutes to 15 minutes.

This process may be repeated as many times as is necessary to remove undesired odors from the sports equipment. However, if the ozone sensor determines that the level of ozone outside the bag (e.g., within the garage, which may be closed) exceeds a pre-determined threshold, the assembly's controller may turn off the ozone generator 200 to prevent the ozone generator 200 from expelling any more ozone. Once the ozone sensor determines that the level of ozone outside the bag is less than a pre-determined threshold value, the controller may again allow the user to turn on the ozone generator 200 to run a deodorizing session.

In certain embodiments, a user may wish to use the assembly away from their home. It should be understood that, in various embodiments, the portable nature of the assembly may allow the assembly to be used away from a user's home (e.g., within a user's car, in the clubhouse of a golf course, or any other suitable location). This may be advantageous in that it may allow a user to deodorize sporting equipment or other items in essentially any suitable location. In particular embodiments, the assembly may include a battery pack that is used to supply power to the ozone generator 200, which may enhance the portable nature of the assembly.

In other embodiments, as may be understood from this disclosure, a user may place the flexible bag containing sports equipment (e.g., or other items) into a second container (which may include, for example, a larger flexible bag or a rigid container such as a bin). The user may then utilize an ozone generator associated with the second container in any suitable manner described herein to cause the ozone generator to pump ozone into the second container. As may be understood by one skilled in the art, the flexible bag (e.g., and its contents) may be exposed to the generated ozone by virtue of being disposed within the second container.

### CONCLUSION

Many modifications and other embodiments of the invention will come to mind to one skilled in the art to which this invention pertains, having the benefit of the teaching presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for the purposes of limitation.

We claim:

1. An assembly for removing odors from clothing or other items, the assembly comprising:
a first container comprising a material that is at least partially ozone impervious and defining a first interior that is accessible via a selectively closeable opening;
an ozone generator that is adapted to generate ozone gas and to expel the ozone gas from the ozone generator through an outlet;
at least one conduit that extends in gaseous communication between the outlet of the ozone generator and the first interior of the first container;
a fastening mechanism for selectively maintaining the selectively closeable opening in a closed orientation in which the fastening mechanism prevents access to the first interior via the selectively closeable opening; and
a second container defining a second interior for at least temporarily storing one or more items for odor removal, wherein:
when the selectively closeable opening is in an open orientation in which the fastening mechanism does not prevent access to the first interior, a user may place the second container into the first interior of the first container via the selectively closeable opening; and
when the second container is in the first interior of the first container, the first interior is in gaseous communication with the second interior allowing for a transfer of at least some ozone gas from the first interior to the second interior.

2. The assembly of claim 1, wherein:
the first container comprises a first flexible bag; and
the second container comprises a second flexible bag.

3. The assembly of claim 2, wherein:
the second flexible bag comprises a material that is at least partially ozone permeable.

4. The assembly of claim 2, wherein:
the second interior of the second flexible bag is accessible via a second selectively closeable opening; and
when the second container is in the first interior of the first container and the second selectively closeable opening is in an open position, the first interior is in gaseous communication with the second interior via the second selectively closeable opening.

5. The assembly of claim 1, wherein;
the at least one conduit comprises tubing that extends from the outlet of the ozone generator to the first interior of the first container;
the tubing is adapted to facilitate the flow of ozone from the outlet of the ozone generator into the first interior of the first container;
the ozone generator is attached adjacent an outer portion of the first container bag; and
an outlet of the tubing is disposed adjacent a central portion of the first interior of the first container.

6. The assembly of claim 1, wherein:
the fastening mechanism comprises:
a toothed zipper having two opposing rows of zipper teeth; and
two sheets of waterproof sheeting that is wrapped around an outer portion of each of the two opposing rows of zipper teeth, wherein:
when the fastening mechanism is in the closed position, the two sheets of waterproof sheeting are configured to squeeze against one another to form a substantially airtight seal along the toothed zipper.

7. The assembly of claim 6, wherein the two sheets of waterproof sheeting comprise fabric-reinforced polyethylene.

8. The assembly of claim 1, wherein:
the first container comprises a rigid bin that:
comprises a locking mechanism and a lid; and
defines an interior chamber accessible via on opening, wherein:
the locking mechanism is configured to maintain the lid adjacent the opening such that the lid prevents physical access to the interior chamber and maintains a seal between the lid and the rigid bin.

9. The assembly of claim 1, wherein the first container is larger than the second container.

10. A method of removing odors from one or more items, the method comprising:
providing a first container comprising a material that is at least partially ozone impervious and defining a first interior that is accessible via a selectively closeable opening, wherein the first container comprises a fastening mechanism for selectively maintaining the selectively closeable opening in a closed orientation in which the fastening mechanism prevents access to the first interior via the selectively closeable opening;
providing an ozone generator that is adapted to generate ozone gas and to expel the ozone gas from the ozone generator through an outlet;
providing at least one conduit;
placing the at least one conduit such that the at least one conduit extends in gaseous communication between the outlet of the ozone generator and the first interior of the first container;
placing one or more items in an interior portion of a second container;
placing the second container within the first container such that the first interior is in gaseous communication with the second interior of the second container;
using the fastening mechanism to maintain the selectively closeable opening in the closed position; and
turning on the ozone generator to cause at least some ozone gas to expel into the first interior portion through the at least one conduit.

11. The method of claim 10, wherein the first container comprises a flexible bag.

12. The method of claim 11, wherein the fastening mechanism comprises:
a toothed zipper having two opposing rows of zipper teeth; and
two sheets of waterproof sheeting that is wrapped around an outer portion of each of the two opposing rows of zipper teeth, wherein:
when the fastening mechanism is in the closed position, the two sheets of waterproof sheeting are configured to squeeze against one another to form a substantially airtight seal along the toothed zipper.

13. The method of claim 10, wherein the two sheets of waterproof sheeting comprise fabric-reinforced polyethylene.

14. The method of claim 10, wherein:
the first container comprises a rigid bin that:
comprises a locking mechanism and a lid; and
defines an interior chamber accessible via on opening, wherein:
the locking mechanism is configured to maintain the lid adjacent the opening such that the lid prevents physical access to the interior chamber and maintains a seal between the lid and the rigid bin.

15. The method of claim 10, wherein:
the ozone generator comprises at least one rechargeable battery configured to provide power to the ozone generator;
the lid comprises at least one solar cell; and
the at least one solar cell is configured to charge the at least one rechargeable battery.

16. The method of claim 10, wherein:
the ozone generator comprises at least one ozone sensor configured to measure a concentration of ozone in an environment in which the ozone generator is located;
the ozone generator is configured to automatically shut off if an ozone concentration measured by the ozone generator is above a particular threshold.

17. The method of claim 16, wherein the particular threshold is 5 parts per million of ozone.

18. The method of claim 10, wherein the first container is large enough to accommodate the second container within the first interior of the first container.

19. The method of claim 10, wherein the first container comprises a car trunk.

20. A method of removing odors from one or more items using an ozone generator, the method comprising:
providing an ozone generator that is adapted to generate ozone gas and to expel the ozone gas from the ozone generator through an outlet;
providing at least one conduit;
placing the at least one conduit such that the at least one conduit extends in gaseous communication between the outlet of the ozone generator and a first interior of a rigid bin, the rigid bin comprising a locking mechanism and a lid and defining an interior chamber accessible via on opening, wherein:
the locking mechanism is configured to maintain the lid adjacent the opening such that the lid prevents physical access to the interior chamber and maintains a seal between the lid and the rigid bin;
placing one or more items in an interior portion of a second container;
placing the second container within the rigid bin such that the first interior is in gaseous communication with the second interior of the second container;
using the fastening mechanism to maintain the selectively closeable opening in the closed position; and
turning on the ozone generator to cause at least some ozone gas to expel into the first interior portion through the at least one conduit.
